(19) United States
(12) Patent Application Publication
(10) Pub. No.: US 2006/0105992 A1
(43) Pub. Date: May 18, 2006

(54) Pharmaceutical formulations of cyclodextrins and selective estrogen receptor modulator compounds

(76) Inventors: Charles Michael Buchanan, Kingsport, TN (US); Norma Lindsey Buchanan, Kingsport, TN (US); Juanelle Little Lambert, Gray, TN (US); Jessica Dee Posey-Dowty, Kingsport, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/268,631

(22) Filed: Nov. 8, 2005

Related U.S. Application Data
(60) Provisional application No. 60/626,004, filed on Nov. 8, 2004.

(51) Int. Cl. A61K 31/724; A61K 31/137; A61K 31/138

(52) U.S. Cl. 514/58; 514/649; 514/651

(57) ABSTRACT

This invention relates to compositions that contain cyclodextrin derivatives and selective estrogen receptor modulators and methods for the production of the compositions of the invention. The invention further relates of methods of administering the compositions of the present invention to a human or animal.

This application claims the benefit of priority of U.S. Provisional Patent Application No. 60/626,004, filed Nov. 8, 2004.

Cyclodextrins (CDs) are cyclic oligomers of glucose, many of which contain 6, 7, or 8 glucose monomers joined by α-1,4 linkages. These oligomers are commonly called α-CD, β-CD, and γ-CD, respectively. Higher oligomers containing up to 12 glucose monomers are also known. Topologically, CDs can be represented as a toroid in which the primary hydroxyls are located on the smaller circumference, and the secondary hydroxyls are located on the larger circumference. Because of this arrangement, the interior of the torus is hydrophobic while the exterior is sufficiently hydrophilic to allow the CD to be dissolved in water. This difference between the interior and exterior faces allows the CD to act as a host molecule and to form inclusion complexes with guest molecules, provided the guest molecule is of the proper size to fit in the cavity. The CD inclusion complex can then be dissolved in water thereby providing for the introduction of a guest molecule that his little or no aqueous solubility into an aqueous environment. Reviews of CD complexes can be found in Chem. Rev., 1997, 97, 1325-1357 and in Supramolecular Chemistry, 1995, 6, 217-223.

Unmodified cyclodextrins, especially β-cyclodextrin, have limited aqueous solubility, have relative large molecular weights, and tend to crystallize from solution. The combination of these issues means that their ability to solubilize and stabilize guest molecules in an aqueous environment is limited. Additionally, unmodified cyclodextrins, e.g. β-cyclodextrin, have been shown to cause renal and liver damage after parenteral administration. These issues have led to exploration of the use of chemically modified or derivatized cyclodextrins that avoid some of these problems. Two examples of derivatized cyclodextrins are hydroxybutenyl cyclodextrins (HBenCD), which are disclosed in U.S. Pat. No. 6,479,467 (2002) and in Carbohydrate Research, 2002, 327(6), 493-507, and sulfonated hydroxybutenyl cyclodextrins (SulfoHBenCD), which are disclosed in U.S. Pat. No. 6,610,671.

Triphenylethylene compounds such as tamoxifen, droloxifene, toremifene, ospemifene, and related structural analogues or metabolites thereof belong to a general class of compounds known as selective estrogen receptor modulators (SERMs). SERMs have the capability of acting as estrogen receptor agonists in some tissues and as antagonists in other tissues. For example, tamoxifen and toremifene are estrogen receptor agonists in bone, the cardiovascular system, and the endometrium, but act as antagonists in breast tissue (Clin Pharmacokinet 2003, 42(4), 361-372). Tamoxifen and toremifene are nonsterodial antiestrogens used clinically as first-line endocrine treatments as well as adjuvant therapy in early and metastic breast cancers in postmenopausal women. Tamoxifen is also approved as a prophylactic agent in women at high risk of developing breast cancer. Patients with estrogen receptor positive cancers respond best to SERMs such as tamoxifen. The preparation of triphenylethylene compounds are disclosed for example in U.S. Pat. Nos. 4,696,949, 5,254,594, and 5,491,173. Triphenylethylene compounds are characterized by having low aqueous solubility which can in turn limit their efficacy. Furthermore, triphenylethylene compounds are known to be unstable due to E-Z isomerization. This isomerization leads to decreased stability and hence, decreased efficacy of pharmaceutical formulations involving triphenylethylene compounds.

### SUMMARY OF THE INVENTION

This invention is directed to compositions comprising a hydroxybutenyl cyclodextrin or derivative thereof and one or more selective estrogen receptor modulators (SERMs). In some embodiments, the SERM is a triphenylethylene compound or a pharmaceutically acceptable salt or base, structural analog or metabolite thereof. In certain embodiments, the triphenylethylene compound is tamoxifen, droloxifene, toremifene, ospemifene or a pharmaceutically acceptable salt or base, structural analog or metabolite thereof.

The hydroxybutenyl cyclodextrin can be hydroxybutenyl-α, β, or γ-cyclodextrin. In other embodiments, the hydroxybutenyl cyclodextrin derivative can be sulfonated hydroxybutenyl-α, β, or γ-cyclodextrin. For example, the hydroxybutenyl cyclodextrin can be hydroxybutenyl-p-cyclodextrin and the hydroxybutenyl cyclodextrin derivative can be sulfonated hydroxybutenyl-β-cyclodextrin.

In certain embodiments, the hydroxybutenyl cyclodextrin has a molar substitution of about 1 to about 12.

In some embodiments, the sulfonated hydroxybutenyl cyclodextrin has a molar substitution of hydroxybutyl sulfonate of about 0.02 to about 7.

In some embodiments, the compositions are dry physical mixtures; in others, the compositions are dry inclusion complexes.

In some embodiments, the compositions are solutions of inclusion complexes in aqueous solution.

In another aspect, the invention relates to methods of increasing the aqueous solubility of a selective estrogen receptor modulator comprising forming a complex or mixture of at least one selective estrogen receptor modulator with a hydroxybutenyl cyclodextrin.

In certain embodiments, the aqueous solubility of the SERM and the cyclodextrin derivative relative to the SERM alone (Sₜₒₜₐₗ/S_{drug}) ranges from 2 to 300; in other embodiments, from 5 to 100; in still other embodiments, from 10 to 50.

In another aspect, the invention relates to methods of increasing the bioavailability of a selective estrogen receptor modulator comprising formulating a selective estrogen receptor modulator with a hydroxybutenyl cyclodextrin. In certain embodiments, the formulation is administered to a subject, such as a human.

Additional aspects and advantages of the invention will be set forth in part in the description which follows, and in part will be obvious from the description, or may be learned by practice of the invention. The embodiments and advantages of the invention can be realized and attained by means of the elements and combinations particularly pointed out in the appended claims.

It is to be understood that both the foregoing general description and the following detailed description are exemplary and explanatory only and are not restrictive of the invention, as claimed. The accompanying drawings, which are incorporated in and constitute part of this specification, and together with the description, serve to explain principles of the invention.

### BRIEF DESCRIPTION OF THE DRAWINGS

FIG. 1 shows the conversion of cyclodextrins to hydroxybutenyl cyclodextrins.

FIG. 2 shows the conversion of hydroxybutenyl cyclodextrins to sulfonated hydroxybutenyl cyclodextrins.

FIG. 3 shows the solubility of toremifene citrate versus HBenβCD and SulfoHBenβCD in unbuffered water.

FIG. 4 shows the solubility of tamoxifen versus HBenβCD in water with an initial pH of 3.

FIG. 5 shows the solubility of tamoxifen versus HBenβCD, SulfoHBenβCD, SBEβCD in phosphate buffered water (pH 3) at different buffering, capacities.

FIG. 6 shows the release of tamoxifen (% maximum) from capsules filled with dry solid inclusion complex at 37° C., pH 6.0.

### DETAILED DESCRIPTION OF THE INVENTION

The present invention may be understood more readily by reference to the following detailed description of the invention and the examples provided therein. It is to be understood that this invention is not limited to the specific methods, formulations, and conditions described, as such may vary. It is also to be understood that the terminology used herein is for the purpose of describing particular aspects of the invention only and is not intended to be limiting.

In order that the present invention may be more readily understood, certain terms are first defined. Additional definitions are set forth throughout the detailed description.

The singular forms “a,” “an,” and “the” include plural referents unless the context clearly dictates otherwise.

The term “hydroxybutenyl cyclodextrin” refers to all forms of hydroxybutenyl cyclodextrins, including hydroxybutenyl-α, ⊕, or γ-cyclodextrins as well as higher oligomers containing up to about twelve glucose monomers. Columns 5-12 of U.S. Pat. No. 6,479,467 disclose the preparation of hydroxybutenyl cyclodextrins and methods of their use; these sections are hereby incorporated by reference. The term “hydroxybutenyl cyclodextrin” also encompasses derivatives thereof, including sulfonated hydroxybutenyl cyclodextrins such as, for example, sulfonated hydroxybutenyl-α, β, or γ-cyclodextrins.

The term “hydroxybutenyl cyclodextrin derivatives” refers to hydroxybutenyl cyclodextrins that have been further elaborated by attachment of substituents to the hydroxyls of the cyclodextrin ring and/or hydroxybutenyl substituent or by manipulation of the olefin of the hydroxybutenyl substituent. Examples of hydroxybutenyl cyclodextrin derivatives include sulfonated hydroxybutenyl-α, β, or γ-cyclodextrins. Columns 5-13 of U.S. Pat. No. 6,610,671 disclose the preparation of hydroxybutenyl cyclodextrin derivatives and methods of their use; these sections are hereby incorporated by reference.

The term “complex” or “inclusion complex” refers to a combination of a chemical compound (such as a drug) and a cyclodextrin wherein the compound or a portion thereof is associated with the cyclodextrin. Typically, the compound, or guest molecule, is included within the cavity of the cyclodextrin, or host molecule, wherein the cavity of the cyclodextrin is the space created by the cyclodextrin torus and the cyclodextrin substituents.

The term “mixture” refers to a combination of a chemical compound (such as a drug) and a cyclodextrin mixed in such a manner that the compound is not substantially included within the cyclodextrin cavity. One example of such a mixture occurs when the compound and the cyclodextrin are physically mixed, for example in a mill or blender. Another example of a mixture is when both the compound and the cyclodextrin are dissolved in a common solvent that will compete with the compound for the cyclodextrin cavity space such that the solvent occupies the cyclodextrin cavity and there is little association of the compound with the cyclodextrin. When a compound-cyclodextrin mixture is placed in a aqueous solvent, such as in a physiological environment, a complex can form in situ provided the rate of complex formation is faster than alternate events such as drug precipitation.

The term “metabolites” refers to compounds (e.g., active species) produced upon introduction of the compounds of the invention into a biological system.

The term “analogs” refers to structurally similar compounds that share at least one biological property.

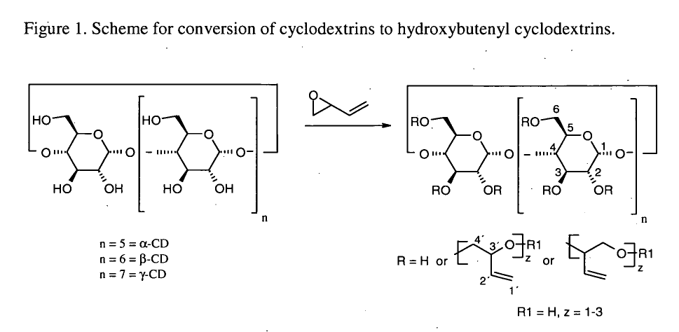

The compositions of the present inventions include a CD or CD derivative and a SERM. The CD or CD derivative is or is derived from a CD of any ring size, including but not limited to α, β, or γ-cyclodextrins. In some embodiments, the CD is a hydroxybutenyl cyclodextrin. In other embodiments, the CD is a hydroxybutenyl-α, β, or γ-cyclodextrins (FIG. 1). In some embodiments, the hydroxybutenyl-β-cyclodextrins have a molar substitution (MS, wherein MS is the total number of substitutents attached to the CD) from about 1 to about 12. In some embodiments, the hydroxybutenyl-β-cyclodextrins are hydroxybutenyl-β-cyclodextrins with a MS from about 3 to about 10. In some embodiments, the hydroxybutenyl-β-cyclodextrins are water-soluble and have a MS from about 4 to about 7. In some embodiments, the hydroxybutenyl-β-cyclodextrins are water-soluble and have a MS from about 4.5 to about 5.5. In some embodiments, the hydroxybutenyl-β-cyclodextrins are water-soluble and have a MS of about 5.

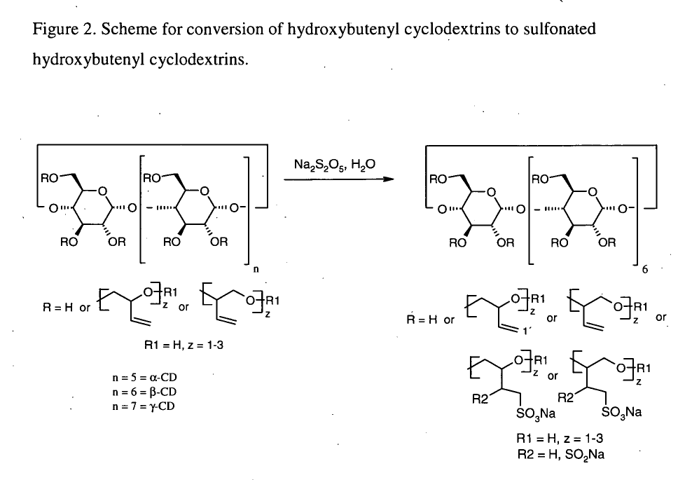

In some embodiments, the hydroxybutenyl cyclodextrin derivatives are sulfonated hydroxybutenyl-α, β, or γ-cyclodextrins (FIG. 2). In some embodiments, the sulfonated hydroxybutenyl cyclodextrins are sulfonated hydroxybutenyl-β-cyclodextrins comprising at least one hydroxybutyl sulfonate substitutent. In some embodiments, the sulfonated hydroxybutenyl-β-cyclodextrins have a MS of hydroxybutyl sulfonate from about 0.02 to about 7. In some embodiments, the hydroxybutenyl-β-cyclodextrins have a MS of hydroxybutyl sulfonate from about 0.05 to about 5. In some embodiments, the hydroxybutenyl-β-cyclodextrins have a MS of hydroxybutyl sulfonate from about 0.1 to about 2. In the case of sulfonated hydroxybutenyl-α, β, or γ-cyclodextrins, those skilled in the art will recognize that these cyclodextrin ethers contain both hydroxybutenyl substitutents and hydroxybutyl sulfonate substitutents. In this case, the total MS is provided by the sum of the hydroxybutenyl MS and the hydroxybutyl sulfonate. In some embodiments, the MS is from about 0.02 to about 12. Cyclodextrin ethers containing at least one hydroxybutyl sulfonate substitutent can also further comprise additional alkyl, sulfinate, or disulfonate substitutents.

The compositions also contain selective estrogen receptor modulators (SERMs). In certain embodiments, the SERM can be a triphenylethylene compound or a benzothiophene compound such as raloxifene. For purposes of example only, the invention is applied to triphenylethylene compounds in the description below. It is to be understood that this description is one non-limiting embodiment of the invention. The compositions and methods of the invention are not limited to triphenylethylene compounds, but are broadly applicable to all SERM compounds.

In some embodiments, the SERMs are triphenylethylene compounds having a structure of Formula I, or a pharmaceutically acceptable salt or metabolite thereof.

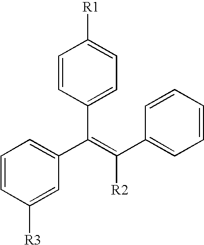

Formula I: Generic structure of triphenylethylenes. It is to be understood that R1, R2, and R3 are simply examples of locations on the triphenylethylene compound upon which derivatization can occur and are not intended to limit the scope of the invention to compounds derivatized at those locations. Thus, triphenylethylene compounds in which derivatization occurs at locations other than R1, R2, and R3 are within the present invention, irrespective of whether such derivatization occurs instead of or addition to derivatization of any or all of R1, R2, and R3. Similarly, the triphenylethylene compound upon which no derivatization occurs is within the present invention. Examples include tamoxifen, wherein R1=OCH₂CH₂N(CH₃)₂, R2=CH₂CH₃, and R3=H, and no additional derivatization exists; toremifene, wherein R1=OCH₂CH₂N(CH₃)₂, R2=CH₂Cl, and R3=H and no additional derivatization exists; ospemifene, wherein R1=OCH₂CH₂OH, R2=CH₂Cl, and R3=H and no additional derivatization exists; and droloxifene, wherein R1=OCH₂CH₂N(CH₃)₂, R2=CH₂CH₃, and R3=OH and no additional derivatization exists. In some embodiments, the triphenylethylene compounds are the free bases of tamoxifen, droloxifene, toremifene, or ospemifene. In some embodiments, the triphenylethylene compounds are pharmaceutically acceptable salts of tamoxifen, droloxifene, toremifene, or ospemifene.

The compositions of the present invention can increase the aqueous solubility of a SERM relative to the aqueous solubility of the SERM in the absence of a CD or CD derivative. The term S_{drug} refers to the intrinsic solubility of the SERM in an aqueous solution and Sₜₒₜₐₗ is the solubility of the SERM in the presence of a CD or CD derivative in the same aqueous solution. The ratio Sₜₒₜₐₗ/S_{drug} indicates the increase in solubility of the SERM in the compositions of the present invention. In some embodiments, Sₜₒₜₐₗ/S_{drug} can range from 2 to 300; in other embodiments Sₜₒₜₐₗ/S_{drug} can range from 5 to 100; in other embodiments Sₜₒₜₐₗ/S_{drug} can range from 10 to 50.

In some embodiments, the triphenylethylene compounds are free bases. In some embodiments, the triphenylethylene compounds are pharmaceutically acceptable salts. In some embodiments, it has been found that free bases of triphenylethylene compounds achieve higher water solubility than the pharmaceutically acceptable salts, thus providing a higher drug loading with increased drug stability.

The pharmaceutically acceptable salts of triphenylethylene compounds are non-toxic salts, such as salts from organic acids (e.g., formic, acetic, propionic, trifluoroacetic, citric, maleic, tartaric, ascorbic, methanesulfonic, benzenesulfonic, toluenesulfonic acids), from inorganic acids (e.g., hydrochloric, hydrobromic, sulfuric, or phosphoric acids), and amino acids (e.g., aspartic or glutamic acids). In some embodiments, the pharmaceutically acceptable salt is a citrate, tartrate, acetate, propionate, mesylate, or HCl salt. The pharmaceutically acceptable salts of triphenylethylene compounds can be prepared by any method, and preparation methods are well known to those skilled in the art. For example, a solution or a suspension of the free base of triphenylethylene compounds can be treated with about one equivalent or slight excess of the pharmaceutically acceptable acid. The resulting salt can then isolated by conventional methods.

Many of the metabolites of triphenylethylene compounds and their pharmaceutically acceptable salts are biologically active. For example, tamoxifen and toremifene undergo phase I metabolism in the liver by microsomal cytochrome P450 enzymes. The major metabolites of tamoxifen are N-desmethyltamoxifen and 4-hydroxytamoxifen. The major metabolites of toremifene are N-desmethyltoremifene and deaminohydroxytoremifene (ospemifene). Both 4-hydroxytamoxifen and deaminohydroxytoremifene -are biologically active SERMS and triphenylethylene compounds of the present invention also include metabolites of triphenylethylene compounds or their pharmaceutically acceptable salts.

In some embodiments, an amount of triphenylethylene compounds or a pharmaceutically acceptable salt or metabolite thereof are used such that the formulation provides the desired therapeutic effect. In some embodiments, they are administered one to four times a day with a unit dosage of 0.25 to 100 milligrams (mg) in human patients. This dosage is varied depending on the age, body weight and medical condition of the patient and the type of administration. One dose of 10-40 mg one time a day is used in some embodiments.

The compositions of the present invention may be in any physical phase, including solid, liquid, and semisolid. Examples of solid compositions include but are not limited to tablets, capsules, or oral powders. In some embodiments, a dry, solid physical mixture of HBenCD or SulfoHBenCD and triphenylethylene compounds or a dry, solid inclusion complex of HBenCD or SulfoHBenCD and triphenylethylene compounds are used, for example to fill a capsule or compressed into a tablet for administration. Dry, solid inclusion complexes are used in some embodiments. Upon exposure to an aqueous environment of use, such as the luminal fluid of the gastrointestinal tract or the salivary fluid of the buccal cavity, the solubility and hence bioavailability of the drug is increased relative to the drug in the absence of HBenCD and SulfoHBenCD.

Liquid formulations include aqueous solutions, and solutions in water soluble organic compounds, or combinations thereof. Examples of water soluble organic compounds suitable for use in the present invention are disclosed in U.S. Patent Application entitled “Cyclodextrin solubilizers for liquid and semi-solid formulations,” filed Nov. 7, 2005, concurrently with this application (no Serial assigned at this time). In some embodiments, aqueous solutions are those in which the water content is at least 20 wt %.

On a weight basis in solid formulations, the ratio of triphenylethylene compound to HBenCD or SulfoHBenCD in some embodiments is from about 1:120 to about 3:1. In some embodiments, the ratio is from about 1:40 to about 2:1. In some embodiments, the molar ratio is from about 1:20 to about 1:1 w/w.

In some embodiments, aqueous solutions comprising HBenCD or SulfoHBenCD, triphenylethylene compounds, and sterile water or other pharmaceutically acceptable aqueous medium are sufficient to form product solutions which can be directly administered, for example parenterally or subcutaneously, directly to human patients. Due the stability provided by HBenCD or SulfoHBenCD, solutions in some embodiments can be stored under appropriate conditions (from about 5° C. to about room temperature) for periods up to 2 years or longer. In some embodiments, an isolated complex can be stored under appropriate conditions at room temperature for periods up to 2 years are longer, and reconstituted into a product solution as needed. The product solution is prepared by dissolving the solid inclusion complex in water or other pharmaceutically acceptable aqueous medium in an amount sufficient to generate a solution of the required strength for oral or parenteral administration.

The compositions of the present invention optionally include additional components. In some embodiments, additional components are useful in achieving or enhancing desired properties of the compositions. Examples of such components include, but are not limited to, fillers, disintegrants, binders, lubricants, dispersing agents, surfactants, thickening agents, as well as other excipients such as cellulose esters and ethers, dyes, and flavorings. Liquid formulations optionally contain buffers, antioxidants, preservatives and tonicity adjusters. Examples of buffers include, but are not limited to, phosphates, acetates, citrates, benzoates, succinates, bicarbonates, and glycine. Examples of antioxidants include ascorbic acid, sodium bisulfite, sodium metabisulfite, monothioglycerol, thiourea, butylated hydroxytoluene, butylated hydroxy anisole, and ethylenediaminetetraacetic acid salts. Preservatives useful in liquid formulations include benzoic acid and its salts, sorbic acid and its salts, alkyl esters of parahydroxybenzoic acid, phenol, chlorobutanol, benzyl alcohol, thimerosal, benzalkonium chloride and cetylpyridinium chloride. The buffers mentioned previously as well as dextrose, glycerin, potassium chloride, and sodium chloride can be used for tonicity adjustment if necessary.

Formulations may contain other excipients known to those skilled in the art such as thickening agents, dispersing agents, dyes, flavorings, buffers, antioxidants, preservatives, and tonicity adjusters. Examples of antioxidants include ascorbic acid, sodium bisulfite, sodium metabisulfite, monothioglycerol, thiourea, butylated. hydroxytoluene, butylated hydroxy anisole, and ethylenediaminetetraacetic acid salts. Preservatives useful in liquid formulations include benzoic acid and its salts, sorbic acid and its salts, alkyl esters of parahydroxybenzoic acid, phenol, chlorobutanol, benzyl alcohol, thimerosal, benzalkonium chloride and cetylpyridinium chloride. Buffers as well as dextrose, glycerin, potassium chloride, and sodium chloride can be used for tonicity adjustment if necessary.

Formulation pH, buffering capacity, and ionic strength are all considered in preparing compositions of the present invention. In the case of triphenylethylene compounds, these are weak electrolytes that can be ionized in appropriate aqueous media. When the triphenylethylene compounds are not ionized, the drug—CD equilibrium is shown by:
Drug+CD⇄Drug:CD.
When the drug is ionized, the drug—CD equilibrium is shown by:
Drug+Drugᵢ+CD⇄Drug:CD+Drugᵢ:CD.
where Drugᵢ is the concentration of the drug in its ionic state. In the case of non-ionized drug, the total solubility in water (S_{water}) is: S_{drug}+S_{complex} where S_{drug} is the solubility of the drug in water and S_{complex} is the solubility of the complex in water. In the case of ionized drug, the total solubility in water is: S_{water}=S_{drug}+S_{drugi}+S_{complex}+S_{compiexi} where S_{drug} and S_{complex} have the same meanings, S_{drugi} is the solubility of the ionized drug in water, and S_{complexi} is the solubility of the complexed ion in water. That is, because of the extra contributions of the ionized species, the total amount of drug solubilized with cyclodextrins with drugs that can be ionized can often be modified. In the absence of cyclodextrin, the intrinsic solubility of a weakly basic drug, such as tamoxifen, in a buffered or pH adjusted aqueous media can be estimated by: Sₜₒₜₐₗ=S_{intrinsic}(1+10^{(pKa-pH)}). That is, the solubility of a weakly basic drug is affected by an order of magnitude for each unit difference between the pKa and the media pH. Hence, the contribution of the drug+ionized drug to the total solubility achieved by complexation with cyclodextrins can be impacted by pH of the aqueous media. This is one example of how manipulating acid strength, buffering capacity, ionic strength, and counterion can influence the amount of triphenylethylene compound solubilized by hydroxybutenyl cyclodextrins in the formulation of the present invention.

pH of the formulation media can be adjusted by any effective agent. Several such agents are known to those skilled in the art, including but not limited to organic acids, organic bases, or buffers. Examples of organic acids include but are not limited to formic, acetic, propionic, trifluoroacetic, citric, maleic, tartaric, ascorbic, methanesulfonic, benzenesulfonic, toluenesulfonic acids. Examples of organic bases include but are not limited to ethylene diamine, triethanolamine, tris(hydroxymethyl)aminomethane, and butyl amine. Examples of buffers include but are not limited phosphates, acetates, citrates, benzoates, succinates, bicarbonates, and glycine. In some embodiments, the concentration of organic acids is from about 0.5 N to about 0.001 N. In some embodiments, the concentration of organic acids is from about 0.2 N to about 0.01 N. In some embodiments, the concentration of organic acids is from about 0.1 N to about 0.05 N. In the case of buffers, the normality in some embodiments is from about 0.5 N to about 0.001. In some embodiments, the normality of buffer is from about 0.1 N to about 0.01. In some embodiments, the normality of buffer is from about 0.05 N to about 0.02. With regard to ionic strength, in some embodiments the ionic strength is less than about 200 mM. In some embodiments, the ionic strength is less than about 100 mM. In some embodiments, increases in ionic strength appear to reduce solubility.

The invention further includes methods of making the compositions of the present invention. Liquid formulations of HBenCD or SulfoHBenCD and triphenylethylene compounds in some embodiments are formed by conventional methods. For example, the desired inclusion complex can be formed in situ by adding a triphenylethylene compound, in an amount less than or equal to the amount corresponding to equilibrium solubility, directly to a solution of HBenCD or SulfoHBenCD in water or other pharmaceutically acceptable aqueous medium. In some embodiments, a dry, solid inclusion complex of HBenCD or SulfoHBenCD and triphenylethylene compounds is formed by the methods of the present invention. In some embodiments, an excess amount of a triphenylethylene compound is added to an aqueous solution of HBenCD or SulfoHBenCD and mixed for a period of time sufficient to obtain equilibrium solubility. Excess drug is removed and the inclusion complex is isolated by drying techniques such as spray drying or freeze drying. In some embodiments, the inclusion complex is isolated by precipitation in a solvent in which the complex has minimal solubility. In some embodiments, dry, solid physical mixtures of HBenCD or SulfoHBenCD and triphenylethylene compounds are formed by any effective method. Examples of such methods include but are not limited to those that provide an intimate physical mixture in which the particle size of the components are reduced. For example, methods such as dry milling can be utilized in the present invention. The molar ratio of the inclusion complex components can vary depending upon the initial solution concentration of each component. In some embodiments, the amount of HBenCD or SulfoHBenCD is such that the molar ratio of triphenylethylene compound to cyclodextrin derivative is from about 1:01 to about 1:30. In some embodiments, the molar ratio is from about 1:0.5 to about 1:10. In some embodiments, the molar ratio is from about 1:1 to about 1:4.

The present invention also includes methods of increasing the bioavailability of a selective estrogen receptor modulator comprising formulating a selective estrogen receptor modulator with a hydroxybutenyl cyclodextrin. In certain embodiments, the formulation is administered to a subject. Suitable subjects include animals, such as mammals and vertebrates. In some embodiments, the subject is a human.

Any method of administration can be used. Examples of such methods include, but are not limited to, oral administration (e.g. buccal or sublingual administration), ingestion through intestinal absorption, anal administration, rectal administration, administration as a suppository, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration at the location of a tumor or internal injury, administration into the lumen or parenchyma of an organ, and parenteral administration. Any technique can be used in the method of administration. Examples of techniques useful in the various forms of administrations above include, but are not limited to, topical application, ingestion, surgical administration, injections, sprays, transdermal delivery devices, osmotic pumps, depositing directly on a desired site, or other means familiar to one of ordinary skill in the art. Sites of application can be external, such as on the epidermis, or internal, for example a gastric ulcer, a surgical field, or elsewhere.

The compositions of the present invention can be applied in any form. Examples include, but are not limited to, creams, gels, solutions, suspensions, liposomes, particles, or other means known to one of skill in the art of formulation and delivery of therapeutic and cosmetic compounds. Some examples of appropriate formulations for subcutaneous administration include but are not limited to implants, depot, needles, capsules, and osmotic pumps. Some examples of appropriate formulations for vaginal administration include but are not limited to creams and rings. Some examples of appropriate formulations for oral administration include but are not limited to: pills, liquids, syrups, and suspensions. Some examples of appropriate formulations for transdermal administration include but are not limited to gels, creams, pastes, patches, sprays, and gels. Some examples of appropriate delivery mechanisms for subcutaneous administration include but are not limited to implants, depots, needles, capsules, and osmotic pumps. Formulations suitable for parenteral administration include but are not limited to aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. Extemporaneous injection solutions and suspensions may be prepared, for example, from sterile powders, granules and tablets.

The invention has been described in detail with particular reference to preferred embodiments thereof, but it will be understood that variations and modifications can be effected within the spirit and scope of the invention. In the drawings and specification, there have been disclosed typical preferred embodiments of the invention. Although specific terms are employed, they are used in a generic and descriptive sense only and not for purposes of limitation, the scope of the invention being set forth in the following claims.

This invention can be further illustrated by the following examples, although it will be understood that these examples are included merely for purposes of illustration and are not intended to limit the scope of the invention.

### EXAMPLES

The following examples are offered for illustrative purposes only.

Hydroxybutenyl cyclodextrins (HBenCD) were prepared according to the general methods described in U.S. Pat. No. 6,479,467. Sulfonated hydroxybutenyl cyclodextrins (SulfoHBenCD) were prepared according to the general methods described in U.S. Pat. No. 6,610,671. Sulfobutyl ether cyclodextrin (SBECD) was prepared according to U.S. Pat. No. 5,376,645. All of the cyclodextrin derivatives were dried at 10-15 mm Hg at room temperature for 14 to 60 h prior to use. All of the drugs were obtained from Apin Chemicals and characterized prior to use.

### Example 1

### The solubility of toremifene citrate in unbuffered water as a function of hydroxybutenyl-β-cyclodextrin and sulfonated hydroxybutenyl-β-cyclodextrin concentration

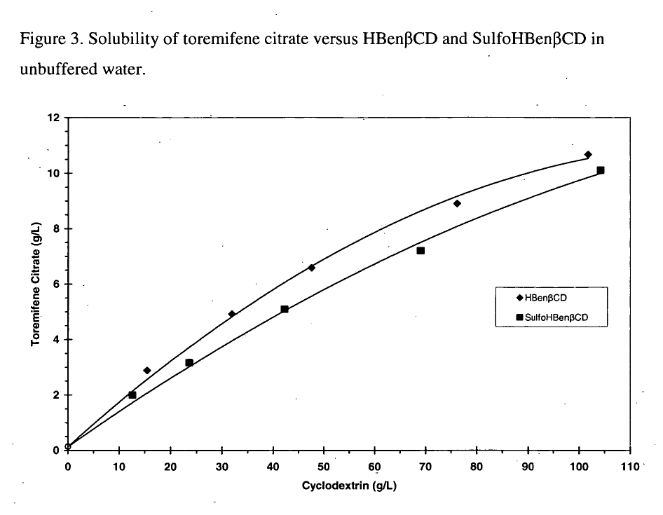

A series of independent HBenβCD_{4.9} (MS=4.9) and SulfoHBenβCD (MS_{sulfonate}=0.3, MS_{butenyl}=4.4) solutions were prepared by accurately weighing known amounts of dry cyclodextrin into volumetric flasks and diluting with unbuffered, deionized water. Unbuffered water was selected on the basis that the drug being evaluated was the citrate salt of toremifene, which has an inherent pH of approximately 3, and no pH adjustment was required for the cyclodextrins being evaluated. Excess toremifene citrate (1^{st} differential scanning calorimetry heating curve Tm=162° C., 2^{nd} DSC heating curve Tg=18° C.) was accurately weighed into 5 mL glass vials with screw caps. To each vial was added HBenβCD_{4.9}, SulfoHBenβCD, or unbuffered water (no CD). The sealed vials were placed on a temperature controlled shaker (23° C.) and the vial contents were mixed at 250 rpm for approximately 60 h. During this period, formation of the toremifene citrate:cyclodextrin inclusion complex equilibrium concentration in water was obtained. The contents of each vial were filtered through a 0.2 micron sterile filter into clean screw cap vials. Each vial was diluted with 1/1 water/ethanol so that the absorbance of the drug was within the linear response range of the UV spectrometer used for determining the concentration of toremifene citrate in each vial. The absorptivity of toremifene citrate was measured at 278 nm. The results from these experiments are illustrated in FIG. 3, which provides a plot of toremifene citrate versus cyclodextrin concentration.

FIG. 3 demonstrates that both HBenβCD and SulfoHBenβCD are very effective at solubilizing toremifene citrate in water. For example, the intrinsic solubility of toremifene citrate in unbuffered water is 0.135 g/L versus 8.903 g/L in the presence of 7.6 wt % HBenβCD, which corresponds to an increase in toremifene citrate solubility of 66×(Sₜₒₜₐₗ/S_{drug}=66).

### Example 2

### The Solubility of Tamoxifen Versus Hydroxybutenyl-β-cyclodextrin Concentration in Water at an Initial pH of 3.0

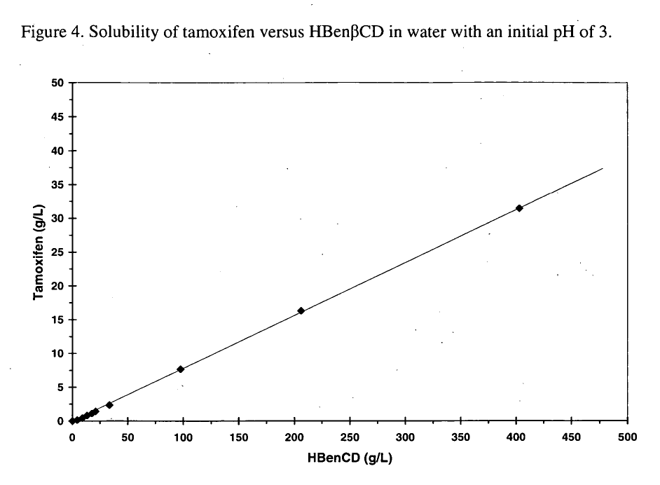

Following the general procedure of example 1, the solubility of tamoxifen in water versus HBenβCD_{4.7} (MS=4.7) concentration was determined. The initial-pH of the HBenβCD_{4.7} aqueous solution was approximately 3.0 and the temperature of the experiment was 25° C. The results are summarized in FIG. 4.

FIG. 4 shows that HBenβCD was extremely effective in solubilizing. tamoxifen in water under these conditions (Sₜₒₜₐₗ/S_{drug}=1533 at 10 wt % HBenβCD). The solubility curve for tamoxifen was linear even at high concentration of HBenβCD. This observation should be contrasted with the observations of Example 1. FIG. 3 demonstrated a concave curvature in the solubility curves with increasing CD concentration. In contrast, no concavity is shown in FIG. 4. This comparison suggests that use of the tamoxifen base may have greater inclusion complex solubility at high complex concentration as compared with the citrate salt, at least under the conditions described in this experiment.

### Example 3

### Solubility of Tamoxifen Versus HBenβCD, SulfoHBenβCD, SBEβCD in Phosphate Buffered Water (pH 3)

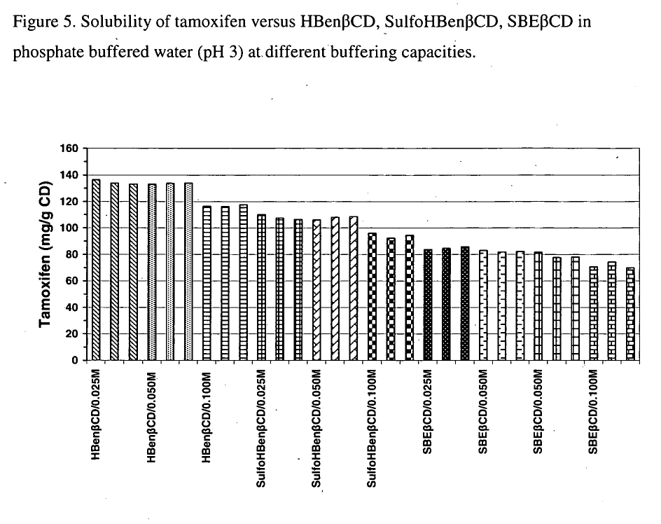

Following the general procedure of example 1, the solubility of tamoxifen in water versus HBenβCD_{4.9} (MS=4.9), SulfoHBenβCD (MS_{sulfonate}=0.3, MS_{butenyl}=4.4), and Sulfobutyl ether cyclodextrin (SBEβCD; MS=7.0) were determined. However, in this experiment the CD concentrations were fixed at 5 wt %. That is, multiple single point determinations in the linear part of the equilibrium solubility curves were collected. The cyclodextrin solutions were made using 3 different phosphate buffers at pH 3 but with differing buffering capacities (0.025 M, ionic strength=22 mM; 0.05 M, ionic strength=24 mM; 0.10 M ionic strength=90 mM). Three separate tests were performed for each combination of CD derivative and buffer. The results are summarized in FIG. 5.

The results show that at a given ionic buffer strength, HBenβCD and SulfoHBenβCD each solubilized more tamoxifen per gram of CD than SBEβCD. With all CDs, the amount of tamoxifen solubilized decreased with increasing buffer ionic strength. For example, 130 mg tamoxifen/g HBenβCD was solubilized in 0.025 M phosphate buffer and 110 mg tamoxifen/g HBenβCD was solubilized in 0.10 M phosphate buffer.

### Example 4

### Preparation of Tamoxifen:HBenβCD Inclusion Complexes

Complex Prepared in Water:

In a 20 mL glass vial with a screw cap, 3 grams (g) of HBenβCD_{4.7} (MS=4.7) were dissolved in 15 mL of unbuffered deionized water. The pH of the HBenβCD_{4.7} aqueous solution was approximately 3.3. To the HBenβCD_{4.7} aqueous solution was added 193 mg of tamoxifen. The vial was briefly vortexed then placed on a roller and the suspension was allowed to mix for approximately 8 days at ambient temperature. The pH of the tamoxifen:HBenβCD_{4.7} aqueous mixture after mixing for this period was approximately 6.5. The tamoxifen:HBenβCD_{4.7} aqueous mixture was then filtered through a 0.45 μm sterile filter into a clean flask for freeze drying. After freeze drying, 3.0268 g of a white tamoxifen:HBenβCD_{4.7} solid complex was obtained. Thermal analysis of this inclusion complex by DSC revealed the complete absence of a melting point for tamoxifen in the 1^{st} heating scan. After cooling from the melt, a Tm for tamoxifen was not observed in a 2^{nd} heating scan. The Tg (2^{nd} heating scan) of HBenβCD₄₇ was observed to drop from approximately 198° C. to 178° C. Proton NMR (DMSO-d₆, 600 MHz) of the tamoxifen:HBenβCD_{4.7} solid was consistent with inclusion complex formation.

This demonstrates the formation of a tamoxifen:HBenβCD_{4.7} inclusion complex in water from which a solid inclusion complex can be isolated.

Complex Prepared in Water/Ethanol:

Following the same general methods used in preparing the tamoxifen:HBenβCD_{4.7} in water, a tamoxifen:HBenβCD_{4.7} complex was prepared in water and ethanol. 6 g of HBenβCD_{4.7} was dissolved in 9 mL of water (pH of the HBenβCD_{4.7} aqueous solution was 2.4). In a separate vessel, 422 mg of tamoxifen was dissolved in 5 mL of EtOH. The solution of tamoxifen in ethanol was added slowly to the HBenβCD_{4.7} aqueous solution. The solution remained clear and no evidence of crystallization or precipitation was observed upon visual inspection after the solution was stored at ambient temperature overnight. After drying, a tamoxifen:HBenβCD_{4.7} solid complex was obtained. Thermal analysis of this inclusion complex by DSC revealed the complete absence of a melting point for tamoxifen in the 1^{st} heating scan. After cooling from the melt, a Tm for tamoxifen was not observed in a 2^{nd} heating scan. The Tg (2^{nd} heating scan) of HBenβCD_{4.7} was observed to drop from approximately 198° C. to 175° C.

This demonstrates that the formation of tamoxifen:HBenβCD_{4.7} mixtures occurs in water/ethanol from which a solid inclusion complex can be isolated by removal of the solvent.

Dissolution Testing:

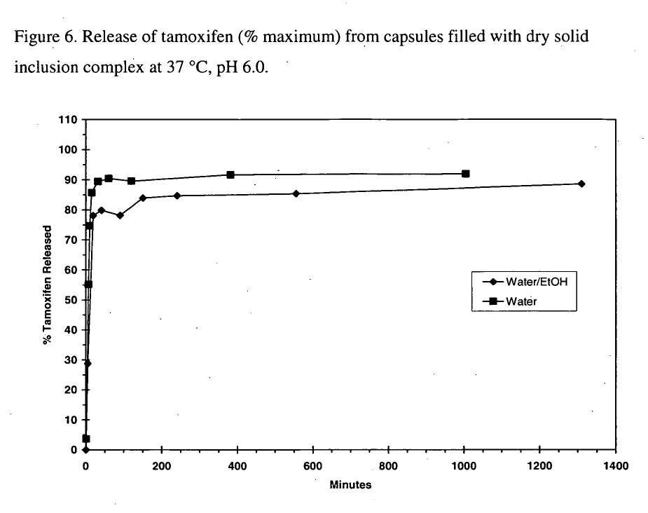

The dry solid inclusion tamoxifen:HBenβCD_{4.7} complexes prepared above were filled into hard shell TORPAC Lock Ring Gel Capsules (Size #0) available from Torpac Capsules Inc., Fairfield, N.J. Dissolution testing was done using a USP #2 apparatus with Teflon coated paddles and 500 ml of a USP pH 6.0 buffer solution. For each experiment the buffer solution was heating to 42° C., followed by vacuum filtration through a 0.45 micron nylon membrane and the vacuum held for an addition 5 minutes. Buffer solution (500 mL) was added to each of the 1000 ML glass dissolution vessels, covered and allowed to equilibrate to 37° C. for 30 minutes. The vessels were kept at constant temperature by a water bath kept at 37° C. The capsules were weighted down with a Varian 3-prong capsule weight. Once the capsules sunk to the bottom of the vessel, the test was initiated by turning the paddles at 100 rpm. The testing was done by withdrawing samples as a function of time with a 10 ml syringe. The removed samples were filtered through a 0.45 micron membrane filter, placed in scintillation vials, and immediately evaluated using a Varian UV-Vis Spectrophotometer, which had been standardized to the concentrations anticipated in the dissolution tests. The samples were measured at 275 nm with a baseline correction from 265-240 nm, using quartz absorption cells. The concentrations measured were then used to calculate the percentage of drug released from the total capsule weight. The results are summarized in FIG. 6.

FIG. 6 shows the dissolution and release of tamoxifen from the capsules at a pH of 6.0 and 37° C. Dissolution and release of the tamoxifen from the capsules was very rapid. In the case of the capsules filed with the inclusion complex prepared in water, approximately 90% of the tamoxifen was released in 30 minutes (approximately 75% at 10 minutes). In the case of the capsules filed with the inclusion complex prepared in water-ethanol, approximately 80% of the tamoxifen was released in 30 minutes increasing to approximately 85% at 150 minutes. Visual inspection revealed no evidence of crystallization or precipitation of tamoxifen during the course of the experiment (approximately 24 h). These observations demonstrate that upon exposure of the filled capsules to a simulated physiological environment, the formulations prepared above both provide for rapid release of the drug and prevent precipitation of the drug over a time period that exceeds the normal intestinal transit time for most humans (8-12 h).

It should be understood that the foregoing relates only to preferred embodiments of the present invention and that numerous modifications or alterations can be made therein without departing from the spirit and the scope of the present invention as defined in the following claims.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many, modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A composition comprising a selective estrogen receptor modulator and a hydroxybutenyl cyclodextrin.

2. The composition of claim 1, wherein the hydroxybutenyl cyclodextrin comprises hydroxybutenyl-β-cyclodextrin.

3. The composition of claim 1, wherein the hydroxybutenyl cyclodextrin has a molar substitution of about 1 to about 12.

4. The composition of claim 1, wherein the hydroxybutenyl cyclodextrin comprises a sulfonated hydroxybutenyl cyclodextrin.

5. The composition of claim 4, wherein the sulfonated hydroxybutenyl cyclodextrin comprises-a sulfonated hydroxybutenyl-β-cyclodextrin.

6. The composition of claim 4, wherein the sulfonated hydroxybutenyl cyclodextrin has a molar substitution of hydroxybutyl sulfonate of about 0.02 to about 7.

7. The composition of claim 1, wherein the selective estrogen receptor modulator comprises a triphenylethylene compound or a pharmaceutically acceptable salt or base, structural analog or metabolite thereof.

8. The composition of claim 7, wherein the triphenylethylene compound comprises tamoxifen, droloxifene, toremifene, ospemifene or a pharmaceutically acceptable salt or base, structural analog or metabolite thereof.

9. The composition of claim 1, wherein Sₜₒₜₐₗ/S_{drug} ranges from 2 to 300.

10. The composition of claim 1, wherein Sₜₒₜₐₗ/S_{drug} ranges from 5 to 100.

11. The composition of claim 1, wherein Sₜₒₜₐₗ/S_{drug} ranges from 10 to 50.

12. A method of increasing the aqueous solubility of a selective estrogen receptor modulator comprising forming a complex or mixture of at least one selective estrogen receptor modulator with a hydroxybutenyl cyclodextrin.

13. The method of claim 12, wherein the hydroxybutenyl cyclodextrin comprises hydroxybutenyl-β-cyclodextrin.

14. The method of claim 12, wherein the hydroxybutenyl cyclodextrin has a molar substitution of about 1 to about 12.

15. The method of claim 12, wherein the hydroxybutenyl cyclodextrin comprises a sulfonated hydroxybutenyl cyclodextrin.

16. The method of claim 15, wherein the sulfonated hydroxybutenyl cyclodextrin comprises a sulfonated hydroxybutenyl-β-cyclodextrin.

17. The method of claim 15, wherein the sulfonated hydroxybutenyl cyclodextrin has a molar substitution of hydroxybutyl sulfonate of about 0.02 to about 7.

18. The method of claim 12, wherein the at least one selective estrogen receptor modulator comprises a triphenylethylene compound or a pharmaceutically acceptable salt or base, structural analog or metabolite thereof.

19. The method of claim 18, wherein the triphenylethylene compound comprises tamoxifen, droloxifene, toremifene, ospemifene or a pharmaceutically acceptable salt or base, structural analog or metabolite thereof.

20. The method of claim 12, wherein Sₜₒₜₐₗ/S_{drug} ranges from 2 to 300.

21. The method of claim 12, wherein Sₜₒₜₐₗ/S_{drug} ranges from 5 to 100.

22. The method of claim 12, wherein Sₜₒₜₐₗ/S_{drug} ranges from 10 to 50.

23. A method of increasing the bioavailability of a selective estrogen receptor modulator comprising formulating a selective estrogen receptor modulator with a hydroxybutenyl cyclodextrin.

24. The method of claim 23, further comprising administering the formulation to a subject.

25. The method of claim 24, wherein the subject is a human.

26. The method of claim 23, wherein the selective estrogen receptor modulator comprises a triphenylethylene compound or a pharmaceutically acceptable salt or base, structural analog or metabolite thereof.

27. The method of claim 26, wherein the triphenylethylene compound comprises tamoxifen, droloxifene, toremifene, ospemifene or a pharmaceutically acceptable salt or base, structural analog or metabolite thereof.
